(19) United States
(12) Patent Application Publication
(10) Pub. No.: US 2004/0146894 A1
(43) Pub. Date: Jul. 29, 2004

(54) Methods of diagnosing and treating stress urinary incontinence

(76) Inventors: Janet A. Warrington, Los Altos, CA (US); Zhaomei Zhang, Sunnyvale, CA (US); Bertha Chen, Menlo Park, CA (US); Yan Wen, San Jose, CA (US); Mary Lake Polan, Palo Alto, CA (US)

Correspondence Address: SPECKMAN LAW GROUP PLLC, 1501 WESTERN AVE, SEATTLE, WA, 98101, US

(21) Appl. No.: 10/684,539

(22) Filed: Oct. 14, 2003

Related U.S. Application Data
(60) Provisional application No. 60/419,007, filed on Oct. 14, 2002.

(51) Int. Cl. C12Q 1/68; C12P 19/34

(52) U.S. Cl. 435/6; 435/91.2

(57) ABSTRACT

The invention disclosed in this application provides methods of diagnosing and treating stress urinary incontinence and predisposition to stress urinary incontinence. These methods are based on the identification of significant differences in gene expression in the pelvic supporting tissues of premenopausal women afflicted with SUI as compared with continent, control women, using microarray-based techniques. The identified differences in gene expression may contribute to altered ECM metabolism and ECM remodeling in pelvic tissue from SUI women. The invention also provides candidate genes for use in diagnosing disorders characterized by pelvic floor dysfunction, including SUI, identification of therapeutic gene targets, evaluation of treatment regimens, prediction of treatment outcome, design of therapeutic agents, and identification of individuals at risk for developing these disorders. Various embodiments of the present invention are disclosed which relate to the above-described uses of candidate genes.

### CROSS-REFERENCE TO RELATED APPLICATIONS

This application claims the benefit of priority to U.S. Provisional application No. 60/419,007, filed Oct. 14, 2002, the entire disclosure of which is incorporated herein by reference.

### STATEMENT OF GOVERNMENT FUNDING

This work was supported by a grant from the National Institutes of Health. The U.S. Government may have certain rights in this invention.

### FIELD OF THE INVENTION

This invention relates to novel methods for diagnosing, preventing and treating stress urinary incontinence. More particularly, the methods of this invention relate to identifying reproductive hormone-dependent differential gene expression in pelvic supporting tissue of premenopausal subjects with stress urinary incontinence (SUI) and subjects predisposed to SUI associated. In one of its embodiments, the methods involve detecting and modulating changes in ECM degradation/remodeling in pelvic supporting tissue. In another embodiment, the invention relates to a novel method of screening for specific modulators and inhibitors of ECM degradation in pelvic supporting tissue.

### BACKGROUND

Pelvic floor dysfunction resulting in SUI is a major health and quality-of-life issue for women in the reproductive and menopausal years. Up to 50% of women over the age of 60 have symptoms of urinary incontinence. In the premenopausal age group, there is a disproportionately higher incidence of urinary dysfunction in women compared to men.

Risk factors that predispose women to the development of stress urinary incontinence include higher parity, pelvic trauma, high body mass index (BMI) and race (see, Benassi et al., (2002), Minerva Ginecol. 54(4): 317-24; Peyrat et al., (2002) BJU Int. 89(1): 61-6; and Graham and Mallett, (2001), Am J Obstet Gynecol. 185(1): 116-20, each of which is incorporated herein by reference). Even in the absence of childbirth, the aging process predisposes to symptoms of incontinence (Buchsbaum et al. (2002), Obstet. Gynecol. 100 (2):226-9, which is incorporated herein by reference).

The pathophysiology of SUI is complex and not well understood. Structurally, the female lower urinary system is supported by pelvic muscles, ligaments, and the bony pelvis, all of which are constantly subjected to stresses, movement, and often trauma. Intact mechanical stability of the lower urinary system is thought to be essential to the continence mechanisms. Defects in any of the support structures may lead to pelvic floor dysfunction.

Some women develop urinary incontinence and/or pelvic floor dysfunction while others with similar obstetrical histories do not. In addition, nulliparous women without any pelvic trauma also develop urinary incontinence and/or pelvic organ prolapse. Epidemiologic data suggest that there are subtle genetic differences in women which predispose some to urinary incontinence and pelvic tissue degradation leading to prolapse.

Damage to the connective tissues, muscle and nerve is thought to result in pelvic floor dysfunction. In the mechanically active environment of the pelvic floor, cells respond to mechanical stimuli by regulation of extracellular matrix structure. Several investigators have documented differences in the connective tissues of women with pelvic floor dysfunction compared to controls. Both collagen content in pelvic ligamentous tissues and its degradative enzymes are altered in tissues from affected women (see, Falconer et al., (1996), Maturitas 24(3):197-204; Rechberger et al., (1998), Am J Obstet Gynecol. 179(6 Pt 1):1511-4; Keane et al., (1997), Br J Obstet Gynaecol. (1997) 104(9):994-8; and Chen et al., (2002), Int Urogynecol J Pelvic Floor Dysfunct. 13(2):80-7; each of which is incorporated herein by reference). Mediators of collagen breakdown, the matrix metalloproteinases and their inhibitors, are also altered in tissues from women with stress urinary incontinence or pelvic floor dysfunction. Ratios of MMP/TIMP mRNAs can be used in the diagnosis of these disorders (see U.S. Pat. No. 6,420,119).

While these data provide insight into the molecular pathophysiology of these disorders, progress has been slow due to the complex extracellular matrix (ECM) interactions that occur simultaneously. In addition, the difficulty of obtaining adequate tissue samples has restricted analysis to only a few enzymes at a time, which is insufficient basis for establishing correlations between molecular pathophysiology and clinical features of the disease. The picture is further complicated by reproductive hormones and growth factors, which modulate the metabolism of extracellular matrix components. Therefore, the expression of enzymes involved in extracellular matrix remodeling may vary according to phase of the menstrual cycle and menopausal status.

In view of the high prevalence rates of stress urinary incontinence among older women, and the absence of a medical therapy for this disorder, there is a continuing need for methods that are capable of detecting predisposition to development of pelvic floor dysfunction leading to stress urinary incontinence. As well, there is a need for methods of screening for therapeutic agents that are capable of reversing the pathophysiologic changes that appear to be associated with collagen and elastin degradation in pelvic tissue of women as they age.

### SUMMARY OF THE INVENTION

The present invention satisfies the above needs by providing methods for diagnosing stress urinary incontinence or predisposition to stress urinary incontinence in a subject. The methods of the present invention are based on the inventors' discovery using oligonucleotide microarray technology that certain classes of genes (including specific ECM genes) are differentially expressed (i.e., up-regulated or down-regulated) in a hormone-dependent manner in pelvic supporting tissues of premenopausal women with SUI by comparison with continent, normal subjects of similar age, parity, body mass index and were in the same stage of the menstrual cycle when tissues were biopsied. Hierarchical clustering analysis provides evidence that differentially expressed gene profiles are capable of discriminating between normal and affected individuals.

In one aspect, the invention provides a method of diagnosing stress urinary incontinence or predisposition to stress urinary incontinence in a subject comprising comparing reproductive hormone-dependent gene expression in a pelvic supporting tissue of the subject and identifying genes that are up-regulated or down-regulated as compared with a predetermined indicator. The predetermined indicator may be empirically determined differential gene expression values obtained by parallel measurements of SUI pelvic supporting tissues and matched tissues of normal controls. Alternatively, the predetermined indicator may be a gene expression profile of the pelvic supporting tissue of the same subject determined at a time prior to the appearance of symptoms of pelvic floor dysfunction.

In a specific embodiment, the method comprises using a nucleic acid array comprising one or more genes selected from the group consisting of genes that are up-regulated during the proliferative phase of the menstrual cycle, genes that are down-regulated during the proliferative phase of the menstrual cycle, genes that are up-regulated during the secretory phase of the menstrual cycle and genes that are down-regulated during the secretory phase of the menstrual cycle. Genes in each of these classes are disclosed in the Detailed Description below.

In a particularly preferred embodiment, the method comprises measuring elafin and alpha1 antitrypsin gene expression in estrogen+progesterone stimulated pelvic supporting tissue from the patient and estrogen stimulated pelvic supporting tissue from the patient and comparing the level of expression with a predetermined indicator.

In yet another aspect, the invention provides in vitro cell based assays which can be used to screen for candidate modulators of gene expression in pelvic supporting tissues, preferably modulators that elevate levels of elastase inhibitors such as elafin and alpha 1 antitrypsin in pelvic supporting tissues of women with SUI and women at risk of developing SUI. Such modulators may include estrogens, antiestrogens, progesterone and antiprogestins, cytokines and growth factors. Therapeutic agents discovered with the use of the screening assays disclosed herein are also intended to be within the scope of this invention.

In still another aspect, the invention provides a method of treating urinary incontinence, preferably in a mammal, and most preferably in a human. The method is intended to be used both for prophylactic therapy (i.e., preventing or delaying the onset of urinary incontinence in predisposed subjects), and for treatment of actual urinary incontinence. The method comprises diagnosing urinary incontinence as described herein and administering agents that reduce proteolysis of ECM components. A preferred method is to administer an effective amount of an elastase inhibitor, a metalloproteinase inhibitor, a modulator of elafin levels, a TIMP or TIMP analog or derivative, a modulator of TIMP levels and combinations thereof. Preferably the active agent or agents are formulated for periurethral or vaginal injection.

### BRIEF DESCRIPTION OF THE FIGURES

FIG. 1. Flow diagram of data reduction process from approximately 6,800 genes to 62 up-regulated and 28 down-regulated genes.

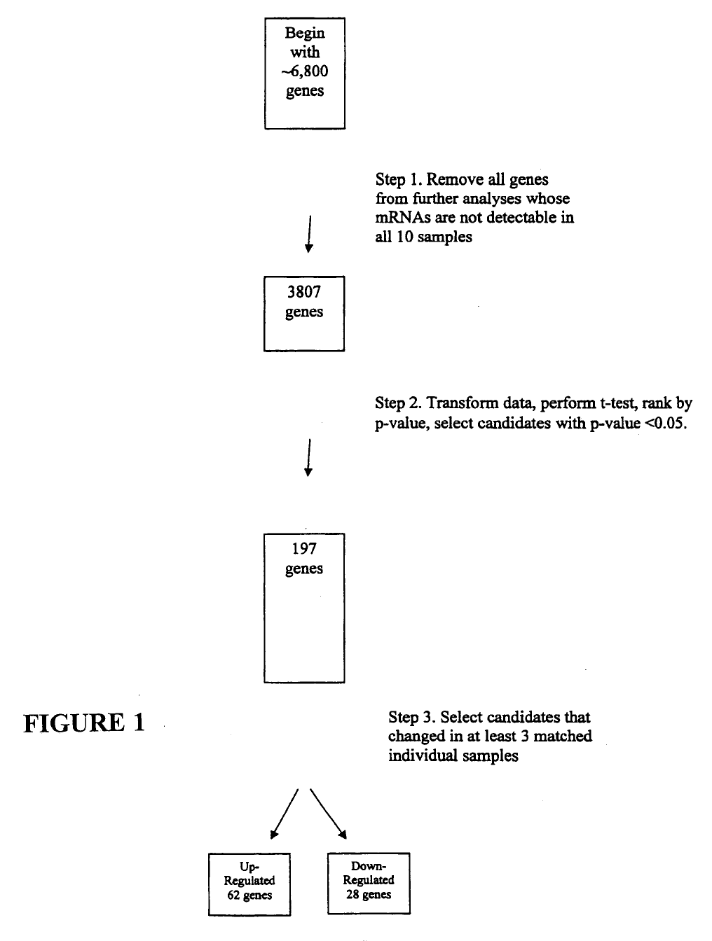

FIG. 2. Comparison of TIMP-1 and estrogen receptor-α mRNA expression in different pelvic tissues within the same individual (L=uterosacral ligament, V=periurethral vaginal mucosa).

FIG. 3. Hierarchical clustering using expression profiles of 90 candidate transcripts using method of cosine correlation of similarity coefficient. SUI and continent control tissue samples clustered independently.

### DESCRIPTION OF THE PREFERRED EMBODIMENTS

A. General Description

The present invention has many preferred embodiments and relies on many patents, applications and other references for details known to those skilled in the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the examples herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press); Stryer, L. (1995) Biochemistry (4^{th} Ed.) Freeman, New York, Gait, “Oligonucleotide Synthesis: A Practical Approach” 1984, IRL Press, London, Nelson and Cox (2000); Lehninger, Principles of Biochemistry 3rd Ed. W. H. Freeman Pub. New York, N.Y.; and Berg et al. (2002) Biochemistry. 5th Ed. W. H. Freeman Pub., New York, N.Y.; all of which are herein incorporated in their entirety by reference for all purposes.

The present invention can employ solid substrates, including arrays in some preferred embodiments. Methods and techniques applicable to, polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760), and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications {Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159, 4,965,188, and 5,333,675, and each of which is incorporated herein by reference in their entirety for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990>>, transcription amplification (Kwoh et al., Proc. Natl. Acad. Sci. USA 86, 1173 (1989) and WO88/10315), self-sustained sequence replication (Guatelli et al., Proc. Natl. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic acid sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel, Methods in Enzymology, Vol. 152, “Guide to Molecular Cloning Techniques” (Academic Press, Inc., San Diego, Calif., 1987); Young and Davism, Proc. Natl. Acad. Sci. USA, 80: 1194 (1983). Methods and apparatus for carrying out repeated and controlled hybridization reactions have been described in U.S. Pat. Nos. 5,871,928, 5,874,219, 6,045,996 6,386,749, and 6,391,623 each of which are incorporated herein by reference.

The present invention also contemplates signal detection of hybridization between ligands in certain preferred embodiments. See U.S. Pat. Nos. 5,143,854, 5,578,832, 5,631,734, 5,834,758, 5,936,324, 5,981,956, 6,025,601, 6,141,096, 6,185,030, 6,201,639, 6,218,803, and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758, 5,856,092, 5,902,723, 5,936,324, 5,981,956, 6,025,601, 6,090,555, 6,141,096, 6,185,030, 6,201,639, 6,218,803, and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and others.

The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent application Ser. Nos. 10/063,559, 60/349,546, 60/376,003, 60/394,574, and 60/403,381.

B. Providing a Nucleic Acid Sample

One of skill in the art will appreciate that it is desirable to have nucleic acid samples containing target nucleic acid sequences that reflect the transcripts of interest. Suitable nucleic acid samples may therefore contain transcripts of interest or nucleic acids derived from the transcripts of interest. As used herein, a nucleic acid derived from a transcript refers to a nucleic acid for whose synthesis the mRNA transcript, or a subsequence thereof, has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like.

Transcripts, as the term is used herein, may include (but are not limited to) pre-mRNA nascent transcript(s), transcript processing intermediates, mature mRNA(s) and degradation products. It is not necessary to monitor all types of transcripts to practice this invention. For example, one may choose to practice the invention by measuring only the mature mRNA levels.

In one embodiment, the sample is a homogenate of cells or tissues or other biological samples. Preferably, the sample is a total RNA preparation of a biological sample. More preferably in some embodiments, the nucleic acid sample is the total mRNA isolated from a biological sample. Those of skill in the art will appreciate that the total mRNA prepared with most methods includes not only the mature mRNA, but also the RNA processing intermediates and nascent pre-mRNA transcripts. For example, total mRNA purified with a poly (T) column contains RNA molecules with poly (A) tails. These poly A+ RNA molecules could be mature mRNA, RNA processing intermediates, nascent transcripts or degradation intermediates.

Biological samples may be any biological tissue or fluid or cells. Frequently the sample will be a “clinical sample”, which is a sample derived from a patient. Clinical samples provide rich sources of information regarding the various states of genetic network or gene expression. Some embodiments of the invention are employed to detect mutations and to identify the function of mutations. Such embodiments have extensive applications in clinical diagnostics and clinical studies. Typical clinical samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

Another typical source of biological samples are cell cultures in which gene expression states can be manipulated to explore the relationships between genes. In one aspect of the invention, methods are provided to generate biological samples reflecting a wide variety of states of the genetic network.

One of skill in the art will appreciate that it is desirable to inhibit or destroy RNase present in homogenates before homogenates can be used for hybridization. Methods of inhibiting or destroying nucleases are well known in the art. In some preferred embodiments, cells or tissues are homogenized in the presence of chaotropic agents to inhibit nuclease. In other embodiments, RNases are inhibited or destroyed by heat treatment followed by proteinase treatment.

Methods of isolating total mRNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993).

In a preferred method, the total RNA is isolated from a given sample using, for example, an acid guanidinium-phenol-chloroform extraction method and polyA+mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)). See also PCT/US99/25200 for complexity management and other sample preparation techniques, which is hereby incorporated by reference in its entirety.

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids to achieve quantitative amplification.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid.

Cell lysates or tissue homogenates often contain a number of inhibitors of polymerase activity. Therefore, RT-PCR typically incorporates preliminary steps to isolate total RNA or mRNA for subsequent use as an amplification template. One tube mRNA capture methods may be used to prepare poly(A)+ RNA samples suitable for immediate RT-PCR in the same tube (Boehringer Mannheim). The captured mRNA can be directly subjected to RT-PCR by adding a reverse transcription mix and, subsequently, a PCR mix. In a particularly preferred embodiment, the sample mRNA is reverse transcribed with a reverse transcriptase and a primer consisting of oligo dT and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template result in amplified RNA. Methods of in vitro polymerization are well known to those of skill in the art (see, e.g., Sambrook, supra).

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

The protocols cited above include methods of generating pools of either sense or antisense nucleic acids. Indeed, one approach can be used to generate either sense or antisense nucleic acids as desired. For example, the cDNA can be directionally cloned into a vector (e.g. Stratagene's p Bluescript II KS (+) phagemid) such that it is flanked by the T3 and T7 promoters. In vitro transcription with the T3 polymerase will produce RNA of one sense (the sense depending on the orientation of the insert), while in vitro transcription with the T7 polymerase will produce RNA having the opposite sense. Other suitable cloning systems include phage lambda vectors designed for Cre-loxP plasmid subcloning (see e.g., Palazzolo et. al., Gene 88: 25-36 (1990)).

Other analysis methods that can be used in the present invention include electrochemical denaturation of double stranded nucleic acids, U.S. Pat. No. 6,045,996 and 6,033,850, the use of multiple arrays (arrays of arrays), U.S. Pat. No. 5,874,219, the use of scanners to read the arrays, U.S. Pat. Nos. 5,631,734; 5,744,305; 5,981,956 and 6,025,601, methods for mixing fluids, U.S. Pat. No. 6,050,719, integrated device for reactions, U.S. Pat. No. 6,043,080, integrated nucleic acid diagnostic device, U.S. Pat. No. 5,922,591, and nucleic acid affinity columns, U.S. Pat. No. 6,013,440. All of the above patents are hereby incorporated by reference in their entireties.

C. Definitions

Array: An array comprises a solid support with peptide or nucleic acid probes attached to said support. Arrays typically comprise a plurality of different nucleic acid or peptide probes that are coupled to a surface of a substrate in different, known locations. These arrays, also described as “microarrays” or colloquially “chips”, have been generally described in the art, for example, U.S. Pat. Nos. 5,143,854, 5,445,934, 5,744,305, 5,677,195, 6,040,193, 5,424,186 and Fodor et al., Science, 251:767-777 (1991), each of which is incorporated by reference in its entirety for all purposes. These arrays may generally be produced using mechanical synthesis methods or light directed synthesis methods which incorporate a combination of photolithographic methods and solid phase synthesis methods. Techniques for the synthesis of these arrays using mechanical synthesis methods are described, for example, in U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are herein incorporated in their entirety for all purposes. Arrays may be packaged in such a manner as to allow for diagnostics or other manipulation of an all inclusive device, see for example, U.S. Pat. Nos. 5,856,174 and 5,922,591, incorporated in their entirety by reference for all purposes. See also U.S. patent application Ser. No. 09/545,207, filed Apr. 7, 2000 for additional information concerning arrays, their manufacture, and their characteristics, which is herein incorporated by reference in its entirety for all purposes.

Physiological state or physiological status: According to the present invention, a physiological state refers to any normal biological state of a cell or organism. The parameters that are considered in determining physiological state include, but are not limited to, age, gender, ethnic origin, and reproductive state, which includes, but is not limited to menstrual state, post-partum, pregnancy, lactation, and nulliparity. For the purposes of this invention the physiological state may be determined by a single indicator. For example, the age of a patient may be the only indicator of physiological state used to categorize a reference sample. Preferably several indicators of physiological state will be used for this purpose. Methods to determine the physiological state of a sample include, but are not limited to, measuring the abundance and/or activity of cellular constituents (expression profile, genotyping), morphological phenotype, or interview of the subject.

Physiological state can refer to, but is not limited to, the physiological state of an organism, an organ, a tissue, a collection of cells or an individual cell. As used herein, physiological state refers to the physiological state of a whole organism or tissue of the organism, e.g., the physiological state of the uterine lining.

Disease state or disease status: In addition to a physiological state, an organism, or tissue or cell of the organism, may or may not be in a disease state. As used in the present application, a disease state refers to any abnormal biological state of an organism or portion thereof. This includes but is not limited to an interruption, cessation or disorder of body functions, systems or organs. In general, a disease state will be detrimental to a biological system. With respect to the present application, any biological state that is associated with a disease or disorder is considered to be a disease state. A pathological state is the equivalent of a disease state.

Disease states can be further categorized into different levels of disease state. As used in the present invention, the level of a disease or disease state is an arbitrary measure reflecting the progression of a disease or disease state. Generally, a disease or disease state will progress through a plurality of levels or stages, wherein the effects of the disease become increasingly severe. The level of a disease state may be impacted by the physiological state of the sample.

Therapy or therapeutic regimen: In order to alleviate or alter a disease state, a therapy or therapeutic regimen is often undertaken. A therapy or therapeutic regimen, as used herein, refers to a course of treatment intended to reduce or eliminate the effects or symptoms of a disease. A therapeutic regimen will typically comprise, but is not limited to, a prescribed dosage of one or more drugs or surgery. Therapies, ideally, will be beneficial and reduce the disease state but in many instances the effect of a therapy will have non-desirable effects as well. The effect of therapy will also be impacted by the physiological state of the organism and by other variables.

Pharmacological state or pharmacological status: Treatment with drugs may affect the pharmacological state of an organism, or sample thereof. The pharmacological state of a sample relates to changes in the biological status following drug treatment. Some of the changes following drug treatment or surgery may relate to the disease state, while others may be unrelated-side effects of the therapy. Some changes will be specific to physiological state. Indicators of pharmacological state include, but are not limited to, the duration of therapy, types and doses of drugs prescribed, degree of patient compliance with a given course of therapy, and/or unprescribed drugs ingested.

Biological state or biological status: According to the present application, the biological state of a sample refers to the state of a collection of cellular constituents, or any other observable phenotype, which is sufficient to characterize the sample for an intended purpose. The biological state of a sample reflects the physiological state, disease state that affects the sample and the pharmacological state, if applicable. Methods to determine the biological state of a sample may include, without limitation, measuring the abundance and/or activity of cellular constituents, assessing morphological characteristics, or a combination of these methods.

The biological status of a sample can be measured or observed by interrogating the abundances and/or activities of a collection of cellular constituents. In various embodiments, the present invention includes making such measurements and/or observations on different collections of cellular constituents

Expression profile: One measurement of cellular constituents that is particularly useful in the present invention is the expression profile. As used herein. An “expression profile” comprises measurement of the relative abundance of a plurality of cellular constituents. Such measurements may include RNA or protein abundances or activity levels. The expression profile can be a measurement for example of the transcriptional state or the translational state. See U.S. Pat. Nos. 6,040,138, 5,800,992, 6,020,135, 6,033,860 and U.S. Ser. No. 09/341,302 which are hereby incorporated by reference in their entireties.

Transcriptional state: The transcriptional state of a sample includes the identities and relative abundances of the RNA species, especially mRNAs present in the sample. Preferably, a substantial fraction of all constituent RNA species in the sample are measured, but at least a sufficient fraction is measured to characterize the state of the sample. The transcriptional state is the currently preferred aspect of the biological state measured in this invention. It can be conveniently determined by measuring transcript abundances by any of several existing gene expression technologies.

Translational state: Translational state includes the identities and relative abundances of the constituent protein species in the sample. As is known to those of skill in the art, the transcriptional state and translational state are related.

The gene expression monitoring system, in a preferred embodiment, may comprise a nucleic acid probe array (such as those described above), membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are expressly incorporated herein by reference. See also Examples, infra. The gene expression monitoring system may also comprise nucleic acid probes in solution.

The gene expression monitoring system according to the present invention may be used to facilitate a comparative analysis of expression in different cells or tissues, different subpopulations of the same cells or tissues, different physiological states of the same cells or tissue, different developmental stages of the same cells or tissue, or different cell populations of the same tissue.

Differentially expressed: The term differentially expressed as used herein refers to up-regulation or down-regulation of the amount of a cellular constituent expressed in one sample relative to another sample (e.g., in an SUI tissue sample relative to a control sample). Differential gene expression can also be used to distinguish between cell types or nucleic acids. See U.S. Pat. No. 5,800,992.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from 20 about 98 to 100%. Alternatively, substantial complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa, Nucleic Acids Res. 12:203 (1984), incorporated herein by reference.

Effective amount refers to an amount sufficient to induce a desired result.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization conditions will typically include salt concentrations of less than about 1M, more usually less than about 500 mM, and preferably less than about 200 mM. Hybridization temperatures can be as low as 5 degrees C., but are typically greater than 22 degrees C., more typically greater than about 30 degrees C. and preferably in excess of about 37 degrees C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridization probes are oligonucleotides capable of binding in a base-specific manner to a complementary strand of nucleic acid. Such probes include peptide nucleic acids. as described in Nielsen et al., Science 254, 1497-1500 (1991), and other nucleic acid analogs and nucleic acid mimetics. See U.S. Pat. No. 6,156,501, filed Apr. 3, 1996. “Hybridizing specifically to” refers to the binding, duplexing, or hybridizing of a molecule substantially to or only to a particular nucleotide sequence or sequences under stringent conditions when that sequence is present in a complex mixture (e.g., total cellular DNA or RNA).

Hybridization probes are oligonucleotides capable of binding in a base-specific manner to a complementary strand of nucleic acid. Such probes include peptide nucleic acids. as described in Nielsen et al., Science 254, 1497-1500 (1991), and other nucleic acid analogs and nucleic acid mimetics. See U.S. Pat. No. 6,156,501, filed Apr. 3, 1996. “Hybridizing specifically to” refers to the binding, duplexing, or hybridizing of a molecule substantially to or only to a particular nucleotide sequence or sequences under stringent conditions when that sequence is present in a complex mixture (e.g., total cellular DNA or RNA).

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferabl, the object species is purified to essential homogeneity (i.e., contaminant species cannot be detected in the composition by conventional detection methods).

Mixed population or complex population: refers to any sample containing both desired and undesired nucleic acids. As a non-limiting example; a complex population of nucleic acids may be total genomic DNA, total genomic RNA or a combination thereof. Moreover, a complex population of nucleic acids may have been enriched for a given population, but include other undesirable populations. For example, a complex population of nucleic acids may be a sample which has been enriched for desired messenger RNA (mRNA) sequences but still includes some undesired ribosomal RNA sequences (rRNA).

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript, or a subsequence thereof, has ultimately served as a template. Detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the cDNA, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides, or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transiently in single-stranded or double-stranded form, including homoduplex, heteroduplex, and hybrid states.

An “oligonucleotide” or “polynucleotide” is a nucleic acjd ranging from at least 2, preferably at least 8, and more preferably at least 20 nucleotides in length, or a compound that specifically hybridizes to a polynucleotide. Polynucleotides as referred to herein include sequences of deoxyribonucleic acid (DNA) or ribonucleic acid (RNA) which may be isolated from natural sources, recombinantly produced or artificially synthesized, and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

A probe is a surface-immobilized molecule that can be recognized by a particular target. Examples of probes include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

A primer is a single-stranded oligonucleotide capable of acting as a point of initiation for template-directed DNA synthesis under suitable conditions (e.g., buffer and temperature) in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with the template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

“Solid support,” “support” and “substrate” are used interchangeably and refer to a material or group of materials having a rigid or semi-rigid surface or surfaces. In many instances, at least one surface of the solid support will be substantially flat, although for certain applications, it may be desirable to physically separate synthesis regions for different compounds with, for example, wells, raised regions, pins, etched trenches, or the like. Solid supports can be in the form of beads, resins, gels, microspheres, or other geometric configurations.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets that can be used in practicing this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term “targets” is used herein, no difference in meaning is intended. A “Probe Target Pair” is formed when two macromolecules have combined through molecular recognition to form a complex.

The term “reproductive hormone” as used herein refers to ovarian steroid hormones, e.g., estrogens and progesterones.

The phrase “hormone-dependent gene expression” as used herein refers to gene expression that is modulated (i.e. increased or decreased) in the presence of a hormone. A combination of hormones may act synergistically, antagonistically, or additively, as these terms are conventionally understood in the context of drug interactions.

D. Menstrual Phase—Differential Gene Expression in Periurethral Vaginal Tissue from Stress Incontinent Women

1. Proliferative (Estrogen) Phase

In the studies described below, gene expression differences were identified by simultaneous measurement of mRNA expression levels of 6,800 genes in periurethral vaginal tissue using microarrays of oligonucleotides. The tissue samples were obtained from age-matched women who were all in the proliferative (estrogen only) phase of the menstrual cycle.

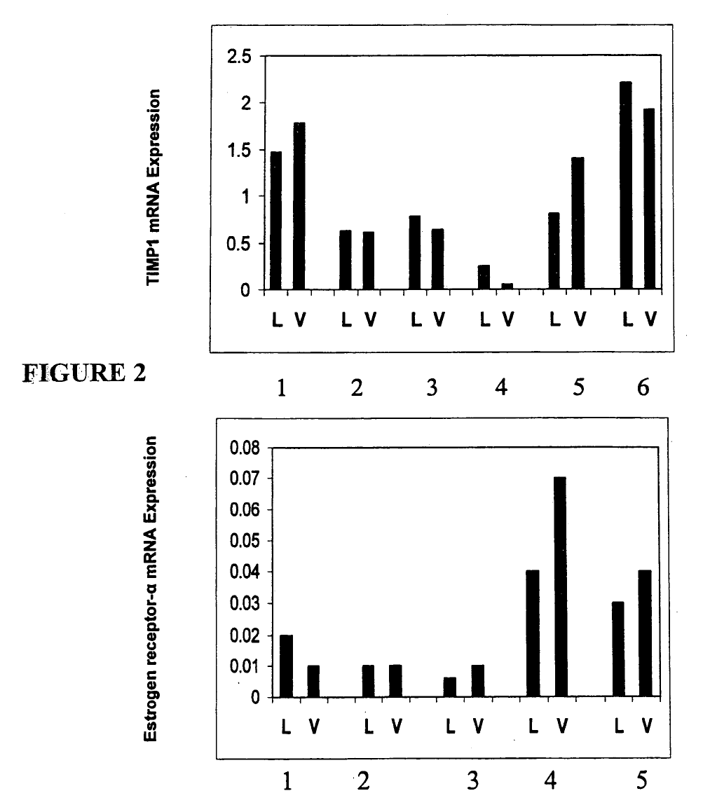

To verify that genetic expressions of pelvic ligamentous tissue is similar to periurethral vaginal tissue within the same individual, we obtained biopies of the uterosacral ligament and periurethral vaginal mucosa from six women and compared TIMP-1 and estrogen receptor-α mRNA expressions in them. Quantitative mRNA expression of TIMP-1 and estrogen receptor-α were almost the same for both types of tissues within each individual in all six women (FIG. 2 and Example 2). Estrogen receptor-α mRNA expression level could not be assessed in one of the women due to small sample size.

Periurethral vaginal tissues were obtained from 11 stress urinary incontinent and 11 age-matched continent, control women undergoing benign gynecologic surgery. Of the 22 samples collected, only 10 samples (5 pairs) met the microarray quality criteria. The average age of the incontinent group was 46.6 years (range 35-54) and 45.8 years (range 45-49) for the continent women. Both groups were also similar in parity and body mass index (Table 1). All ten women were premenopausal and in the proliferative phase of the menstrual cycle as determined by uterine histology. Pelvic organ prolapse was no greater than stage I in any of the ten women, as identified by POP-Q (Bump et al, (1996), Am J Obstet Gynecol. 175(1): 10, which is incorporated herein by reference).

Of the 90 candidate genes identified in this study identified as differentially expressed with a p value assessed by two independent methods as <0.05, 62 were up-regulated and 28 were down-regulated (Table 2). The average fold increase in the up-regulated genes from SUI women compared to controls ranged from 1.3 to 4.8, while that of the down-regulated genes ranged from 1.2 to 3.1-fold.

Genes in the up-regulated group include TGF-beta3, extracellular matrix molecules (e.g., laminin and collagen type VI), and myocyte function-related proteins (e.g. LIM protein and dystrophin). Transforming growth factor beta3 (TGF-beta3) is a cytokine involved in cell growth regulation and differentiation, stimulation of extracellular matrix, and modulation of immune responses. It appears to increase production of collagen type I and type III mRNA levels (see Lee and Nowak, (2001), J Clin Endocrinol Metab. 86(2): 913-20, which is incorporated herein by reference), which are the principal components providing tensile strength to ligamentous tissues. Of the extracellular matrix proteins found to be up-regulated, laminin regulates the assembly of collagens into high-order fibrils in connective tissues and has been identified as a candidate gene in the pathogenesis of certain types of Ehlers-Danlos syndrome and other connective tissue disorders (see, Jepsen et al., (2002), J Biol Chem 277(38): 35532-35540, which is incorporated herein by reference), suggesting the possibility of weaker ligamentous fibrils in SUI women. Not only were the above collagen metabolism genes altered, but smooth muscle function proteins were also found to be differentially expressed in SUI women compared to continent controls. Dystrophin, which was up-regulated by 1.35-fold, is a gene primarily expressed in smooth muscle. Mutations in this gene can lead to muscular dystrophies (see, Roberts and Dickson, (2002), Curr Opin Mol Ther, 4(4):343-8, which is herein incorporated by reference).

Down-regulated genes that may participate in collagen metabolism include: laminin-related protein (LamA3), BPI80 (collagen XVII), serine/threonine protein kinase, type II interleukin-1 receptor, and PDGF-associated protein. Type II interleukin-1 receptor can attenuate the effects of interleukin-1 with respect to induction of inflammatory mediators, matrix metalloproteinase activity, and proteoglycan synthesis, (see, Amin, Clin Orthop., (2000), (379 Suppl): S179-88, which is incorporated herein by reference), while PDGF-associated protein is thought to be involved in proteolysis and protease inhibition (see, Seidel et al. (1998), Brain Res Mol. Brain Res. 60(2): 296-300, which is incorporated herein by reference). Thus, down-regulation of these genes in some embodiments may be indicative of increased extracellular matrix degradation. Serine/threonine protein kinase is reportedly regulated by TGF-beta 1, and may contribute to enhanced matrix formation during fibrosis (see, Fillon et al., (2002), Cell Physiol Biochem. 12(1): 47-54, which is incorporated herein by reference).

Genes that mediate metalloproteinase activity were shown to be differentially expressed. This may reflect the insidious progression of pelvic floor dysfunction. Defects in pelvic floor support take many decades to become evident, unlike aggressive disease growth processes such as cancer, where gene expressions differ drastically, which may explain the relatively small average-fold change in gene expression (circa 1.3 to 4.8) compared to the several orders of magnitude changes seen in cancers.

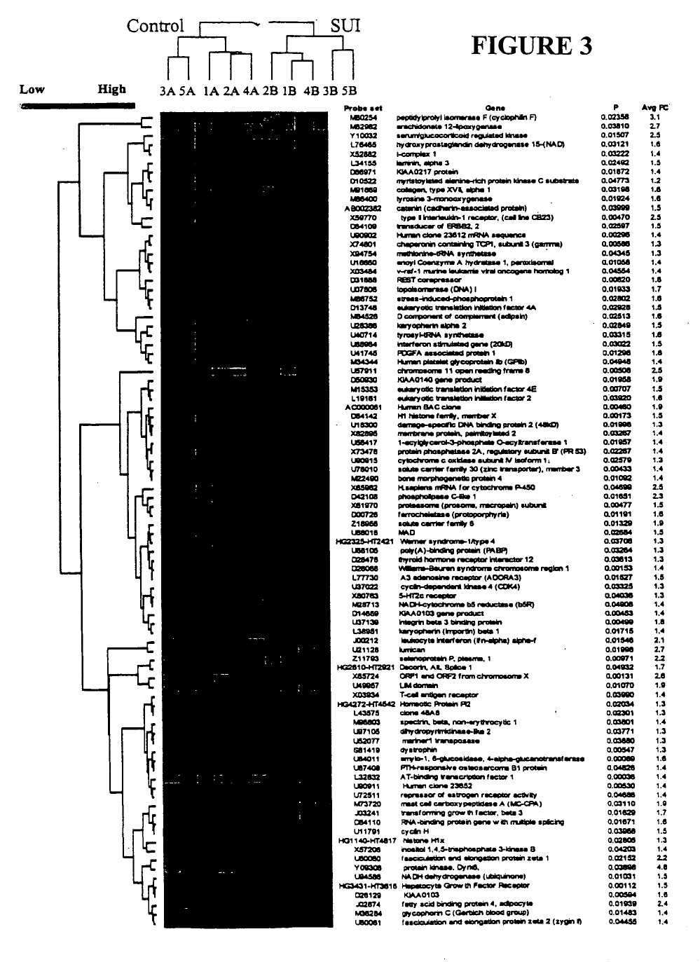

Clustering analysis was applied to assess the ability of the 90 up- and down-regulated candidate genes in SUI to discriminate between normal and affected individuals (FIG. 3). In two-dimensional analyses, gene expression patterns that are similar group together, i.e., cluster. Consequently, one expects that tissues with markedly different expression patterns would form distinct clusters when sorted by expression pattern in all five tissue pairs. The clustering results support the importance of these genes for distinguishing between SUI and control tissue biological activity and may reflect complex genetic variations that are predictive of future development of incontinence.

2. Secretory (Estrogen+Progesterone) Phase

In the studies described below, microarrays of oligonucleotides were used as gene-specific probes to simultaneously measure the quantitative expression of approximately thirty-three thousand genes in periurethral vaginal tissue obtained from 5 premenopausal women with SUI and 5 continent, control women who were matched for age, parity and body mass index. All 10 women were in the secretory (estrogen+progesterone) phase of the menstrual cycle, as determined by endometrial histology. Vaginal wall tissues from 14 additional women with SUI and 9 control women were used for the QC-PCR and Western blot analyses (see Examples 2 and 3 below). The demographics of these women are simlar to the five pairs of women examined in the microarray experiments.

Using the raw data, without any statistical analysis, we identified 20 genes that are up-regulated in all five pairs of women with SUI compared to controls having fold-changes ranging from 1.2 to 78.8. The number of differentially expressed genes increased to 58 up-regulated and 36 down-regulated when we catalogued changes occurring in at least four out of five pairs. From this list, up-regulated genes that appear to function in ECM metabolism include skin-derived protease inhibitor 3(elafin), IL-1 receptor antagonis (IL-1RA), keratin 6, keratin 14, keratin 16 and psoriasin 1. Down-regulated genes include alpha 2 actin, actin depolymerizing factor, smooth muscle myosin, myosin light chain kinase, receptor (calcitonin) activity modifying protein 1 (RAMP1), tropomyosin 1, microfibril-associated glycoprotein=2, insulin-like growth factor binding protein 7, and collagen type IV alpha chain. From this list, we chose elafin (up-regulated in 5 out of 5 pairs), IL-1RA (up-regulated in 4 out of 5 pairs) and RAMP1 (down-regulated in 4 out of 5 pairs) for QC-PCR and Western blot confirmation (Examples 2 and 3 below). These proteins were selected because of their relatively large fold changes (greater than 2). The differential gene and protein expressions of these genes were confirmed by both methods, as well as by immunofluorescent cell staining in fibroblasts cultured from anterior vaginal wall tissues.

MAS5 and RMA algorithms were used to normalize raw data, and the normalized data were then subjected to various statistical analyses (see Example 1 below). 387 differentially expressed genes were detected with MAS5 and 480 genes with RMA. Seventy nine genes were identified by both methods as significant differentially expressed genes. Elafin, keratin 16, collagen type XVII and plakophilin were consistently identified as up-regulated genes by both MAS5 and RMA. Elafin (a serine protease inhibitor), keratin 14 and keratin 16 were consistently up-regulated by 16-, 5- and 6-fold, respectively.

Elafin is an epithelial host-defense protein that is absent in normal skin but highly induced in keratinocytes in skin affected by psoriasis, in epidermal skin tumors, and after wound healing. Hyperproliferation is a functional physiologic response to wound healing. Other genes whose expression is known to be highly up-regulated in healing include cytokeratins 6, 16 and 17. The expression of elafin is tightly connected to abnormal epithelial differentiation and hyperproliferation, as are keratins 6,16 17. Thus far, elafin gene expression has been examined in keratinocytes, breast and lung epithelial cell lines. Our study is the first to document elafin expression in pelvic fibroblasts.

The identification of increased expression of elafin and keratin 16, both involved in the hyperproliferative response, points to altered cellular responses in pelvic tissues from women with SUI. Furthermore, elastin metabolism is implicated since elafin is a serine protease inibitor involved in the elastin degradation pathway. A decrease in the serine protease inhibitor alpha-1 antitrypsin mRNA and protein expression in vaginal tissues from women with SUI compared to controls in the proliferative phase of the menstrual cycle was also documented in our studies. These results suggest that both collagen and elastin metabolism may be altered in women with SUI/pelvic floor dysfunction and that differential activation of genes involved in these pathways is hormone-dependent. Currently there is only one other published study that used microarray-based technology to investigate pelvic floor dysfunction. Visco et al. examined gene expression in pelvic muscle (pubococcygeus) muscle) in postmenopausal women with prolapse (stage III or IV) and in control subjects without prolapse and observed differential expression of genes involved in muscle and ECM metabolism.

The differences in gene expression disclosed herein may contribute to altered ECM metabolism and ECM remodeling in pelvic tissue from SUI women. The identified genes are candidate genes for use in diagnosing disorders characterized by pelvic floor dysfunction, including SUI, identification of therapeutic gene targets, evaluation of treatment regimens, prediction of treatment outcome, designing drugs, and for identifying individuals at risk for developing these disorders. Various embodiments of the present invention relate to the above-described uses of candidate genes. Different families of genes may be modulated by the hormonal fluctuations of the menstrual cycle, and it is possible that collagen and elastin metabolism are further modulated by the interplay between different hormonal milieu over several decades.

### EXAMPLES

These Examples are presented to illustrate the practice of various embodiments of the invention and are not intended to limit the scope of the invention as claimed.

### Example 1

### Analysis of Differential Gene Expression in Pelvic Supporting Tissue Using Oligonucleotide Microarrays

Patient Selection

Subjects were premenopausal women undergoing benign gynecologic surgery with no history of endometriosis, gynecologic malignancies, pelvic inflammatory conditions, connective tissue disorders, emphysema or prior pelvic surgery. Continent women were considered as controls while women undergoing surgery for urinary incontinence were identified as cases. The diagnosis of urinary incontinence was confirmed by urodynarnic studies. The subjects were matched for age, parity and body mass index. The degree of pelvic organ prolapse in both groups of subjects was no greater than stage I as determined by POP-Q. The study was approved by the Institutional Review Board of Stanford University Medical School. Informed consent of the subjects was obtained for excision of pelvic supporting tissue used in this study.

Tissue Collection and RNA Isolation

Approximately 1 cm² of full-thickness, peri-urethral vaginal wall was excised 1 cm lateral to the urethrovesical junction identified by a Foley balloon, from women undergoing surgery for stress urinary incontinence. A 0.5 cm² sample of uterosacral ligament was also obtained from six participants for comparison studies between different pelvic tissues. Smaller, 0.5 cm² biopsies of vaginal wall from a similar area were excised in continent, control women undergoing benign gynecologic surgeries. For analysis, tissue samples were selected from subjects in the proliferative (estrogen only) phase of the menstrual cycle and from subjects in the secretory (estrogen plus progesterone) phase of the menstrual cycle. Tissue specimens were frozen in liquid nitrogen immediately after excision.

Total RNA was extracted with TRIZOL reagent (Gibco BRL Life Technologies, Grand Island, N.Y.) according to the protocol suggested by the manufacturer. At least 30 μg total RNA was extracted from the tissue, and a portion was subjected to gel analysis to verify the integrity of the RNA.

Extraction. Amplification, and Labeling of mRNA

Extraction of total RNA, amplification, and labeling of mRNA were carried out as previously described (Mahadaveppa and Warrington, (1999) Nat. Biotechnol. 17 (11): 1134-6) and as published in the GeneChip® Expression Technical Manual (Affymetrix, Inc., Santa Clara, Calif.).

Sample quality was assessed using four criteria: the appearance of 18S and 28S rRNA by agarose gel electrophoresis; an A260/A280 spectrophotometric ratio less than or equal to 2; a GAPDH (glyceraldehyde-3-phosphate dehydrogenase) 3 prime to 5 prime ratio less than 3; and a minimum of 26% transcripts detected as present calls in each sample.

Labeled target was fragmented by incubation at 94° C. for 35 min in the presence of 40 mM Tris-acetate, pH 8.1, 100 mM potassium acetate, and 30 mM magnesium acetate. The hybridization solution consisted of 20 μg fragmented cRNA and 0.1 mg/ml sonicated herring sperm DNA in 1× MES buffer (containing 10 mM MES, 1M Na+, 20 mM EDTA, and 0.01% Tween 20).

Hybridization, subsequent washing, and staining of the arrays were carried out as outlined in the GeneChip® Expression Technical Manual (Affymetrix, Inc) on Affymetrix arrays. HuGene F1 arrays were used for analysis of gene expression in proliferative phase tissue samples and Human Genome U133A oligonucleotide chip sets were used for analysis of gene expression of secretory phase tissue samples. The arrays were synthesized as described previously using light-directed combinatorial chemistry (Fodor et al., (1993) Nature 364 (6437): 555-6), which is incorporated herein by reference).

Following washing and staining, probe arrays were scanned twice (multiple image scan) at 3 μm resolution using the GeneChip® System confocal scanner made for Affymetrix by Hewlett-Packard, Inc., Palo Alto, Calif.) and GeneChip® Scanner 3000.

Microarray Data Analysis

GeneChip® 5.0 Software (Affymetrix, Inc.) was used to analyze the scanned images and to obtain probe usage and quantitative information. Data were filtered to remove from further analysis probe sets (PM, perfect match; MM, mismatch) that were not detectable in all of the samples. The images were analyzed to determine an intensity value for each probe set within each gene represented on the array. The MAS 5 default algorithm (Affymetrix Microarray suite 5.1©) defines an average difference for each probe set on each array using a log₂ transformed probe intensities to correct for nonspecific binding. For analysis of data generated with U133A arrays, in addition to the MAS 5 algorithm, we used the Robust Multiarray Average (RMA) algorithm developed by Rafael Irizarray (Department of Biostatistics, Johns Hopkins University) for background correction and quantile normalization for normalizing the probe intensities from different arrays to the same distribution. An R package with these algorithms is available for downloading from the Bioconductor project Web site at www.bioconductor.org.

Microarray data was subjected to statistical analysis using multiple methods to identify genes that were differentially expressed in SUI subjects as compared with continent controls. These methods included: simple t tests of both parametric and non-parametric formulations, SAM (Statistical Analysis of Microarrays, Tusher et al, (2001), Proc Natl Acad Sci USA 98: 5116-5121), PAM (Prediction Analysis for Microarrays, Tibshirani et al, (2002) Proc Natl Acad Sci USA 99: 6567-6572) and empirical Bayes (Efron B et al., (2001) Journal of the American Statistical Association, 96: 1151-1160). We calculated test statistics using both the raw data (parametric) and their ranks (non-parametric). The calculated p values were then adjusted according to different multiple testing procedures and cut-off points were set to select genes that showed significant differences in differential expression at different levels. For example, in the SAM analysis, we used the permuted version of parametric t tests. A number of genes were selected according to according to a pre-set false detection rate (FDR). Using PAM, we identified a list of significant genes remaining by setting a shrinking parameter. In the empirical Bayes method, we calculated the posterior probability and selected a list of genes by controlling the Bayesian FDR. A common list of genes was then generated from these methods.

Hierarchical Clustering

Hierarchical clustering analysis was performed with data obtained from tissue samples taken during the estrogen phase of the menstrual cycle to assess the ability of the 90 up- and down-regulated candidate genes in SUI to discriminate between normal and affected individuals. A matrix based ward clustering analysis employing the Cosine correlation of similarity coefficient was performed using GeneMaths software (Applied Maths, Kortrijk, Belgium). The results shown in FIG. 3 suggest that the analyzed genes may be important for distinguishing SUI and control pelvic supporting tissue phenotypes and may reflect complex genetic variations which may be predictive of the development of incontinence in individuals who have not yet developed the clinical symptoms of this condition.

### Example 2

### Quantitative Competitive PCR

Quantitative competitive PCR was used to quantitate levels of expression of TIMP-1 and estrogen receptor-α in pelvic ligamentous tissues and periurethral vaginal mucosa of SUI and continent, control subjects in order to confirm the similarity of gene expression in different pelvic supporting tissues. Quantitative competitive PCR was also used to quantitate levels of elafin, IL-1 receptor antagonist (IL-1 RA) and receptor activity modifying protein 1 (RAMP-1), which were representative of genes that showed significant differential expression in comparisons of SUI and continent, normal subjects in the microarray experiments described in Example 1 above.

Primers for Reverse Transcription (RT) and PCR

Specific sequences of oligonucleotide primers for TIMP-1, estrogen receptor-a elafin, TL-1 ra and RAMP-1 were obtained from Gene Bank Database of the National Center for Biotechnology Information (NCBI) of the National Institutes of Health (at www.2.ncbi.nlm.nih.gov/cgi-bin/genbank). Corresponding sets of primers were found with the help of the program OLIGO 5.0 Primer Analysis Software (National Bioscience, Plymouth, Minn.) and were synthesized by the “protein, aminoacid and nucleic acid-(PAN) facility,” Beckman Center, Stanford University, Stanford, Calif. The human β-actin primers that were used to amplify an external standard were obtained from C lontech Laboratories Inc., Palo Alto, Calif. β-actin mRNA expression was employed as an external positive control, being detected in all the samples studied, thus confirming the integrity of the RNA and the RT-PCR process.

For RT-PCR, the Gen Amp RNA PCR kit (Perkin-Elmer, Foster City, Calif.) was used. Nineteen microliters of RT-MasterMix for each sample were prepared containing 5 mmol/L MgCl₂, 1× PCR buffer II, 1 mmol/L of each deoxy-NTP, 2.5 μl/L oligo (deoxythimidine)₁₆, 20 IU ribonuclease inhibitor (all Perkin-Elmer), and 100 IU Moloney murine leukemia virus reverse transcriptase (Gibco BRL), and 1 μg total RNA diluted in 1 μl DEPC-treated H₂O and placed into a 0.5 ml thin wall PCR tube (Applied Scientific, South San Francisco, Calif.). RT-MasterMix in PCR tubes was covered with 50 μl of light white mineral oil (Sigma, St. Louis, Mo.) and kept on ice until the RT. RT was carried out in the DNA Thermal Cycler 480 (Perkin-Elmer) using a program with the following parameters: 42° C., 15 min; 99° C., 5 min; then quenched at 4° C. After the reaction was completed, samples were stored at −20° C. until the PCR. As negative control, 1 μl DEPC-treated H₂O without RNA sample was subjected to the same RT reaction.

Construction of the Competitive- and Target-cDNA Fragment

A 484 base pair (bp) fragment of native estrogen receptor-α, a 288 bp fragment of native TIMP-1 c-DNA, a 455 bp fragment of elafin, 422 bp fragment of IL-1 RA cDNA, and 446 bp fragment of RAMP-1 (i.e., the target) was obtained by PCR amplification of reverse-transcribed total RNA from vaginal mucosa with the regular 3′ and 5′ primers. The PCR product was visualized by agarose gel electrophoresis stained with ethidium bromide (ETB), and the cDNA was extracted from the gel, purified with an agarose gel extraction kit (Amersham Pharmacia Biotech Ltd., Cambridge, UK), and quantitated by spectrophotometry (Pharmacia Biotech Ltd., Cambridge, UK). To construct a competitive cDNA fragment, a floating primer with a sequence complementary to the cDNA between the 3′ and 5′ primer binding sites was designed by attaching the complementary sequence of the binding site of the original 3′-end estrogen receptor-a, TIMP-1, elafin, IL-1RA or RAMP-1 primer. After PCR with the regular 5′-end primer and the 3′-end floating primer, the PCR product was visualized by agarose gel electrophoresis stained with ETB, and cDNA extraction, purification and determination of the concentration were performed as described above. This step resulted in cDNA fragments of estrogen receptor-a (302 bp), TIMP-1 (124 bp), elafin (278 bp), IL-1 RA(155 bp) and RAMP-1 (240 bp).

Quantitative Competitive-PCR

The standard curve for TIMP-1, estrogen receptor-α, elafin, IL-1 RA and RAMP-1 was constructed by co-amplification of a constant amount of competitive cDNA (0.1 attomol for TIMP-1, 0.01 attomol for estrogen receptor-α, and 1 attomol each for elafin, IL-1RA and RAMP-1), with declining amounts of target cDNA (4.6-0.004 fmol for estrogen receptor-α, and 0.3-0.625 fmol for the other targets) obtained by serial dilution. Sixty microliters of the cDNA mix were added to 40 μl PCR-MasterMix containing 1.9 mmol/L MgCl₂ solution, 10× PCR buffer II, 0.2 mmol/L of each deoxy-NTP, 2.5 U Taq polymerase (Perkin-Elmer Corp., Foster City, Calif.), corresponding paired primers in a concentration of 0.2 μmol/L of each primer to a total volume of 100μ, and 14.5 μl DEPC-treated H₂O. The reaction was covered with 50 μl light white mineral oil and put in the Perkin-Elmer DNA Thermal Cycler 480. PCR cycles were started at 95° C. for 5 min to denature all proteins; 30 cycles for 45 seconds at 94° C.; 45 seconds at 55° C.; 60 seconds at 72° C. The reaction was terminated at 72° C. for 5 min and was quenched at 4° C. Two percent agarose gel (Gibco BRL, Gaithersburg, Md.) electrophoresis was carried out in an H5 electrophoresis chamber. Gels were stained with ethidium bromide (Sigrna). Aliquots (25 μl) of each PCR product and dye buffer were analyzed in parallel with a 100-bp DNA ladder (Gibco BRL, Gaithersburg, Md.) as a 35 standard. After completion of electrophoresis, the gel blot was analyzed, and photocopies of the blot were printed by UV densitometry (Gel-Doc 1000 system, Bio-Rad Laboratories, Inc., Hercules, Calif.). The logarithmically transformed ratios of target cDNA to competitive cDNA were plotted against the log amount of initially added target cDNA in each PCR to obtain the standard curve. This standard curve was highly reproducible and linear. The values obtained from the regression line of the standard curve (y=b+mx) allowed us to calculate the amount of cDNA transcripts in an unknown sample. Competitive cDNA was added to each unknown sample before PCR (0.01 finol estrogen receptor, 0.1 fmol TIMP-1, 1 attomol elafin, 1 attomol IL-1RA, 1 attomol RAMP-1). The ratios of the densities of sample target cDNA TIMP-1, estrogen receptor-α, elafin, IL-1RA and RAMP-1 (484 bp, 228 bp, 455 bp, 422 bp, 446 bp, respectively) to competitive cDNA (302 bp, 1245 bp, 278 bp, 155 bp, and 240 bp, respectively) were logarithmically transformed and compared with the values obtained from standard curves.

QC-PCR results confirmed that these genes were differentially expressed and, in addition, showed that gene expression in pelvic ligamentous tissue and periurethral vaginal tissue within a given individual was quantitatively similar (FIG. 2).

### Example 3

### Western Blot Analysis

Western blot analysis was used to confirm protein expression of two up-regulated (elafin, IL-1 RA) and one down-regulated (RAMP-1) gene. Twenty micrograms of total protein from each patient was separated by 10% SDS-PAGE under reducing conditions and blotted onto nitrocellulose membranes (Pierce, Rockford, Ill.) in an electrophoretic transfer cell (Bio-Rad, Hercules, Calif.). Blots were blocked with 5% non-fat milk at 4° C. overnight. After blocking, the membrane was washed three times in PBST (PBS, pH 7.4 and 0.1% Triton). The membrane was incubated in 1:100 dilution of polyclonal antibody to human elafin (SKALP) (Cell Science, Norwood, Mass.),m 1 μg/ml monoclonal anti-human IL-1RA antibody (R&D Systems, Minneapolis, Minn.), or 1:200 dilution of rabbit polyclonal antibody against RAMP1 (Santa Cruz Biotechnology, Inc., Santa Cruz, Calif.) for 1 hour at room temperature, followed by 3 washes in PBST. The membrane was then incubated in 1:50,000 dilution of sheep anti-mouse IgG or 1:5000 dilution of donkey anti-rabbit IgG (Amersham Pharmacia Biotech) conjugated to HRP for 1 hour at room temperature, followed by three washes in PBST. Blots were developed by chemiluminescence. The results of this analysis indicated that these proteins were differentially expressed.

### Example 4

### Immunofluorescence Cell Staining

Immunofluorescence cell staining was carried out to verify that the identified proteins were expressed in pelvic fibroblasts. Fibroblasts from vaginal wall tissues were cultured in a 4-well chamber slide. The cells were fixed with 4% PFA and treated with 5% Triton. After washing with TBS-T and blocking with 5% normal goat serum and 1% BSA, the slides were incubated with 50 μg/ml of of rabbit anti-IL-1RA (R&D System, Minneapolis, Minn.) or 1/50 goat anti-elafin (Cell Sciences, Norwood, Mass.) or mouse anti-vimentin (Chemicon, Temecula, Calif.) primary antibody at 4° C. overnight. After washing, the slides were incubated with goat anti-mouse IgG-TRITC and goat anti-rabbit IgG-FITC (secondary antibodies for vimentin and IL-1RA respectively) or goat anti-mouse IgG-TRITC and donkey anti-goat-FITC (secondary antibodies for vimentin and elafin respectively) at room temperature for 1 hour in a dark chamber. After washing and mounting, the slides were examined with a fluorescent microscope. The results confirmed the expression of these proteins in pelvic fibroblasts. This is believed to be the first to document elafin expression in pelvic fibroblasts.

### Example 5

### In Vitro Screening Assays for Candidate Modulators

In vitro cell based assays are useful in screening for candidate modulators of gene expression in pelvic supporting tissues. Of particular interest in this regard are modulators that elevate levels of elastase inhibitors such as elafin and alpha 1 antitrypsin in pelvic supporting tissues of women with SUI and women at risk of developing SUI. As described in this application, elastase-mediated tissue proteolysis is believed to be an important component of pelvic tissue dysfunction and SUI. It is believed that the use of these assays will reveal candidate modulators that can preferably be administered to patients by periurethral or vaginal injection.

Fibroblast Cultures

Fibroblast cultures are started from biopsy specimens of vaginal tissue using a known explant method. Ten small tissue samples of approximately 1 mm³ are placed into 25 cm² of untreated plastic tissue culture flasks for primary explantation. After allowing tissue fragments to attach to the plastic surface for 15 minutes, 5 ml of culture medium consisting of 90% DMEM/10% fetal bovine serum, is added to the flasks. Cultures are incubated at 37° C. in an atmosphere of 95% air and 5% CO₂ and experiments performed on confluent cultures between passages 3 and 7.

Tests of Putative Modulators

Time course studies of both mRNA and protein expression in fibroblast cell cultures are carried out to determine an appropriate timeframe for these experiments which is expected to be 24 hours. Our results show that alpha 1 antitrypsin expression is down-regulated in SUI during the estrogen phase of the menstrual cycle and that elafin expression is up-regulated during the estrogen+progesterone phase of the cycle. This suggests that estrogen, progesterone, antiestrogen and antiprogestin may modulate the expression of these genes. Dose response studies will be carried out with each of these agents, singly and in combination. In addition, TGF-β and other cytokines and growth factors involved in ECM remodeling will be tested.

After 24 hours of incubation, cell supernatants are isolated and concentrated and fibroblast monolayers are homogenized using Triton X-100. Total elastase activity is measured by methods that are well known in the art, and elastase activities in supernatants and cell homogenates are examined by gel electrophoresis with zymography to identify the type(s) of elastase enzymes whose activity is modulated. Replicate cultures are harvested for analysis of mRNA levels by QC-RT-PCR as described elsewhere in this application.

### Example 6

### Drug Screening Assay

Fibroblast cultures from vaginal cuff tissue of premenopausal women with and witlout stress incontinence are established in untreated plastic dishes as described above and in the scientific literature. Between passages 3 and 7, confluent cultures from each group of women are treated for an appropriate length of time with increasing concentrations of a putative therapeutic agent, for example, the Roche MMP inhibitor RS113,456). A suitable dosage range for this inhibitor is 1 μg/ml-100 μg/ml in DMEM. Initial experiments are performed to determine the time course for inhibition of MMP activity by RS113,456. After addition of RS113,456 to fibroblast cultures, the earliest period at which steady state MMP inhibition is reached is identified. That time is subsequently used as a standard time of incubation for dose response curves. Cell supernatants are isolated and concentrated and fibroblast monolayers are homogenized using Triton X-100. To measure the specific proteolytic inhibition produced by RS113,456, the MMP activities in supernatants and cell homogenates are examined by zymography with electrophoresis on premade gelatin gels (BioRad, Hercules, Calif.) (50,51). Using antibody which recognizes the COL2-3/4Cₛₕₒᵣₜ epitope, the level of collagenase activity is determined directly.

In like manner, putative agonists of ECM enzyme inhibitors can be tested for therapeutic efficacy.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

All of the publications, patent applications and patents cited in this application are herein incorporated by reference in their entirety to the same extent as if each individual publication, patent application or patent was specifically and individually indicated to be incorporated by reference in its entirety.

We claim:

1. A method of diagnosing stress urinary incontinence or predisposition to stress urinary incontinence in a premenopausal subject comprising analyzing reproductive hormone-modulated gene expression in a pelvic supporting tissue of the subject and comparing the obtained results with a predetermined indicator of differential expression of said genes in SUI subjects relative to normal continent subjects.

2. The method of claim 1, wherein said tissue is obtained from the subject during the proliferative (estrogen only) phase of the menstrual cycle.

3. The method of claim 1, wherein said tissue is obtained from the subject during the secretory (estrogen+progesterone) phase of the menstrual cycle.

4. The method of claim 1, wherein the pelvic supporting tissue is selected from the group consisting of vaginal cuff tissue, periurethral vaginal wall tissue, vaginal epithelium, periuterine tissue and pelvic ligamentous tissue.

5. The method of claim 1, wherein gene expression is assessed by measuring the levels of gene transcription in said tissue sample.

6. The method of claim 5, wherein gene expression is measured by quantitative competitive PCR.

7. The method of claim 5, wherein gene expression is measured by hybridization of gene transcripts from said tissue to oligonucleotide probes.

8. The method of claim 1, wherein gene expression is analyzed using a nucleic acid array.

9. The method of claim 1, wherein the predetermined indicator comprises empirically determined differential gene expression values obtained by parallel measurements of SUI pelvic supporting tissues and matched tissues of normal controls.

10. The method of claim 1, wherein the predetermined indicator comprises a gene expression profile of the pelvic supporting tissue of the same subject determined at a time prior to the appearance of symptoms of pelvic floor dysfunction.

11. The method of claim 1, wherein gene expression is analyzed for one or more genes selected from the group consisting of genes involved in ECM metabolism, collagen degradation, elastin degradation and myocyte function.

12. The method of claim 8, wherein said array comprises one or more gene probes selected from the group consisting of elafin, IL-1RA, keratin 14, keratin 16, collagen type XVII, plakophilin, RAMP1, and alpha 1 antitrypsin.

13. The method of claim 8, wherein said array further comprises one or more gene probes selected from the group consisting of alpha 2 actin, actin depolymerizing factor, smooth muscle myosin, myosin light chain kinase, tropomyosin, tropomyosin 1, microfibril-associated glycoprotein-2, insulin-like growth factor binding protein 7 and collagen type IV alpha chain.

14. The method of claim 8, wherein said array comprises one or more gene probes selected from the group consisting of TGF-beta 3, laminin, collagen type VI, LIM protein, distryophin, laminin-related protein (LAMA3), collagen XVII (BP180), serine/threonine protein kinase, type II interleukin-1 receptor, PDGF-associated protein, matrix metalloproteinases, and alpha 1 antitrypsin.

15. A method for treating a premenopausal patient diagnosed as having urinary incontinence or predisposition to urinary incontinence, according to the method of claim 1, the method comprising reducing proteolysis of collagen and elastin in pelvic supporting tissue of the patient and determining the effect of the treatment on the patient's condition.

16. The method of claim 15, wherein reducing proteolysis of collagen and elastin in pelvic supporting tissue is accomplished by administering to the patient an effective amount of one or more of the following:
(a) an elastase inhibitor;
(b) a metalloproteinase inhibitor;
(c) a modulator of elafin levels;
(d) a TIMP or a TIMP analog or derivative;
(e) a modulator of TIMP levels
